(19) United States
(12) Patent Application Publication
(10) Pub. No.: US 2013/0236548 A1
(43) Pub. Date: Sep. 12, 2013

(54) Method for treating cancer by using Fe-based particles

(71) Applicant: NATIONAL CHENG KUNG UNIVERSITY, Tainan City (TW)

(72) Inventors: Dar-Bin SHIEH, Tainan City (TW); Li-Xing YANG, New Taipei City (TW); Ya-Na WU, Yunlin County (TW)

(73) Assignee: NATIONAL CHENG KUNG UNIVERSITY, Tainan City (TW)

(21) Appl. No.: 13/795,640

(22) Filed: Mar. 12, 2013

Related U.S. Application Data
(60) Provisional application No. 61/609,436, filed on Mar. 12, 2012.

(51) Int. Cl. A61K 9/14; A61K 33/26

(52) U.S. Cl. CPC A61K 9/14; A61K 33/26; Y10S 977/773; B82Y 5/00
USPC 424/489; 424/646; 977/810; 977/915; 977/773

(57) ABSTRACT

A method for treating a cancer is disclosed, which comprises: administering an effective amount of Fe-based particles to a subject in need, wherein the Fe-based particles have core-shell structures. Herein, each Fe-based particle of the present invention comprises: an Fe elemental core with zero valent irons; and a covering layer formed on partial or whole surface of the Fe elemental core, wherein a material of the covering layer is a metal, a metal doped with dopants, a metal alloy, a polymer, carbon, a metal oxide or a nonmetal oxide, and the shape of the Fe-based particles is a rod, a sphere, a cubic or a dumbbell, with the proviso that the metal is not Au.

### CROSS REFERENCE TO RELATED APPLICATION

This application claims the benefit of filing date of U.S. Provisional Application Ser. No. 61/609,436, entitled “Application of zero-valent iron based nanoparticle or clusters in tumor therapeutics” filed Mar. 12, 2012 under 35 USC §119(e)(1).

### BACKGROUND OF THE INVENTION

1. Field of the Invention

The present invention relates to methods for treating cancer and, more particularly, to methods for treating cancer by using Fe-based particles with zero valent irons.

2. Description of Related Art

Foods or food additives, and environmental pollutions have been a source of contention as a cause or catalyst for promoting cancer in recent years. Not coincidentally, the same event is happening as well in the developed countries and around the world, positing as an alarming sign that the incidence rates of cancers are quite high. According to the data published by the American Cancer Society, cancer is being proved to be the most significant threat to public health.

The general methods for treating cancer include surgery, radiotherapy, chemotherapy and immune therapy. In recent years, the development of several therapeutic agents has led to cancer treatments through new anti-cancer mechanisms, and it has been proven that the survival rate of patients can be increased by treating them with these therapeutic agents. Generally, the therapeutic agents can treat cancers through inhibition of cell cycle progression, angiogenesis, farnesyl transferase, and tyrosine kinases. Although it is known that certain agents exhibit therapeutic effects on cancer, these agents do have their limitations. For example, the commercial chemical agents kill not only tumor cells but also normal cells. Hence, it is desirable to provide novel anti-cancer agents which can only kill tumor cells but keep normal cells survive.

On the other hands, nanoparticles have unique electrical, chemical, physical and optical properties due to its size associated effects. Organic or inorganic nanoparticles have been applied as carriers to transport and deliver drugs or genes into target organs or cells. Alternatively, some nanoparticles could convert externally applied energy to therapeutics such as hyperthermia, free radical generation and ionic radiation. Since the nanoparticles have the aforementioned properties, the applications thereof can further be increased if it is improved that the nanoparticles has anti-cancer properties.

### SUMMARY OF THE INVENTION

The object of the present invention is to provide a method for treating a cancer, wherein the Fe-based particles used therein can selectively kill tumor cells without significant cytotoxicity to normal cells.

To achieve the object, a first aspect of the method for treating the cancer of the present invention comprises: administering an effective amount of Fe-based particles to a subject in need, wherein the Fe-based particles have core-shell structures. Herein, each Fe-based particle comprises: an Fe elemental core with zero valent irons; and a covering layer formed on partial or whole surface of the Fe elemental core, wherein a material of the covering layer is a metal, a metal doped with dopants, a metal alloy, a polymer, carbon, a metal oxide or a nonmetal oxide, and the shape of the Fe-based particles is a rod, a sphere, a cubic or a dumbbell, with the proviso that the metal is not Au.

Furthermore, a second aspect of the method for treating the cancer of the present invention comprises: administering an effective amount of Fe-based particles to a subject in need, wherein each Fe-based particles is an Fe elemental particle with zero valent irons.

In the method for treating the cancer of the present invention, the Fe-based particles comprise the Fe elemental cores or the Fe elemental particles with zero valent irons, which can be used as a self-detoxification anti-cancer drug. When the Fe-based particles of the present invention are applied to the cancer treatment, an effective amount thereof can selectively kill tumor cells and inhibit the growth of the tumor cells. In addition, the Fe-based particles of the present invention can selectively kill tumor cells without significant cytotoxicity to normal cells. Hence, when the Fe-based particles are applied on tumor therapies, the side effect thereof can further be reduced. In addition, the Fe-based particles of the present invention can also be used as diagnostic reagents, such as imaging agents for Magnetic Resonance (MRI) or Computed Tomography (CT). Hence, when the Fe-particles are applied to the subject in need, the tracing object of the Fe-particles can further be achieved. Therefore, the Fe-based particles of the present invention have dual-functions of anti-cancer drugs and imaging agents.

In the first aspect of the method of the present invention, the material of the covering layer is Ag, Pt, a metal oxide, an AgPt alloy, an AgAu alloy, or a PtAu alloy. Preferably, the material of the covering layer is Ag or ferric oxide (Fe₂O₃).

In addition, in the first aspect of the method of the present invention, a size of each Fe-based particle can be in a nano- or submicro-scale. For example, the size of each Fe-based particle is in a range from about 5 nm to about 5 μm. Preferably, the size of each Fe-based particle is in a range from nm to 1 μm. More preferably, the size of each Fe-based particle is in a range from 5 nm to 50 nm.

Furthermore, in the first aspect of the method of the present invention, a size of the Fe elemental core in each Fe-based particle can be in a nano- or submicro-scale. For example, the size of each Fe elemental core is in a range from about 5 nm to about 5 μm. Preferably, the size of the Fe elemental core is in a range from 5 nm to 1 μm. More preferably, the size of the Fe elemental core is in a range from 5 nm to 50 nm.

In addition, in the first aspect of the method of the present invention, the covering layer can be formed on partial or entire surface of the Fe elemental core in each Fe-based particle. Preferably, the covering layer is formed on the entire surface of the Fe elemental core. Herein, a thickness of the covering layer in each Fe-based particle can be in a nano-scale. Preferably, the thickness of the covering layer is in a range from about 0.7 nm to about 6 nm.

In the second aspect of the method of the present invention, a size of each Fe-based particle can be in a nano- or submicro-scale. For example, the size of each Fe-based particle is in a range from about 5 nm to about 5 μm. Preferably, the size of each Fe-based particle is in a range from 5 nm to 1 μm. More preferably, the size of each Fe-based particle is in a range from nm to 50 nm.

In the first and second aspects of the method of the present invention, the cancer can be any types of cancers generally known in the art, such as bladder cancer, bone cancer, brain cancer, breast cancer, cervical cancer, colon cancer, endometrial cancer, esophageal cancer, leukemia, liver cancer, lymphoma, kidney cancer, osteosarcoma, ovarian cancer, pancreatic cancer, prostate cancer, skin cancer including basal and squamous cell carcinoma and melanoma, small intestine cancer, stomach cancer, thymus cancer and thyroid cancer, but the scope of applicability of the present invention is not limited thereto. Preferably, the cancer is an oral cancer, which may be classified into various histological types such as teratoma, adenocarcinoma derived from a major or minor salivary gland, lymphoma from tonsillar or other lymphoid tissue, or melanoma from the pigment-producing cells of the oral mucosa.

In addition, in the first and second aspects of the method of the present invention, the subject can be mammalian. Preferably, the subject is human.

Except for the aforementioned aspects of the method for treating cancers, the present invention further provides a pharmaceutical composition for tumor therapeutics, which comprises an effective amount of the aforementioned Fe-based particles; and a pharmaceutically acceptable carrier.

The Fe-based particles and the pharmaceutical composition for treating cancers of the present invention can be administered via parenteral, inhalation, local, rectal, nasal, sublingual, or vaginal delivery, or implanted reservoir. Herein, the term “parenteral delivery” includes subcutaneous, intradermic, intravenous, intra-articular, intra-arterial, synovial, intrapleural, intrathecal, local, and intracranial injections.

According to the pharmaceutical composition of the present invention, the term “pharmaceutically acceptable carrier” means that the carrier must be compatible with the active ingredients (and preferably, capable of stabilizing the active ingredients) and not be deleterious to the subject to be treated. The carrier may be at least one selected from the group consisting of active agents, adjuvants, dispersants, wetting agents and suspending agents. The example of the carrier may be microcrystalline cellulose, mannitol, glucose, non-fat milk powder, polyethylene, polyvinylprrolidone, starch or a combination thereof.

In addition, the term “treating” used in the present invention refers to the application or administration of the Fe-based particles or the pharmaceutical composition containing the Fe-based particles to a subject with symptoms or tendencies of suffering from cancer in order to cure, heal, alleviate, relieve, alter, remedy, ameliorate, improve, prevent or affect the symptoms or tendencies of cancers. Furthermore, “an effective amount” used herein refers to the amount of each active ingredients such as the Fe-based particles required to confer therapeutic effect on the subject. The effective amount may vary according to the route of administration, excipient usage, and co-usage with other active ingredients.

Other objects, advantages, and novel features of the invention will become more apparent from the following detailed description when taken in conjunction with the accompanying drawings.

### BRIEF DESCRIPTION OF THE DRAWINGS

FIG. 1 is a result showing the cytotoxicity of Fe-based particles according to Embodiment 1 of the present invention;

FIG. 2 is a result showing the cytotoxicity of Fe-based particles according to Embodiment 2 of the present invention;

FIG. 3 is a result showing the cytotoxicity of Fe-based particles according to Embodiment 3 of the present invention; and

FIG. 4 is a result showing the cytotoxicity of Fe-based particles with Fe elemental cores and Au sells.

### DETAILED DESCRIPTION OF THE PREFERRED EMBODIMENT

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

### [Material and Method]

### Embodiment 1

### Preparation of Fe-Based Particles with Zero Valent Irons

To synthesize the Fe-based particles, 20 mL 1-octadecene and 0.3 mL oleylamine was mixed at 120° C. for 30 min in argon. Later, 0.7 mL iron pentacarbonyl was added to the 1-octadecene and oleylamine mixture at 180° C. for 20 min with continuously argon condition. The solution was cooled down to 160° C. before the further addition of 0.15 mL iron pentacarbonyl. Then, the solution was aged at 160° C. for 30 min with the presence of 0.3 ml oleic acid (1 mM). The particles were then washed with hexane and ethanol, and then kept in argon prior to the use.

Here, the Fe-based particles were placed in desiccated inert gas to prevent Fe being oxidized. The Fe-based particles also can be placed in a liquid with low reactivity such as oxygen-depleted alcohol to prevent oxidation.

After the aforementioned process, the Fe-based particles were obtained, which are Fe elemental particles with zero valent irons. Herein, the size of each Fe-based particle is about 15.34±1.39 nm, and the shape thereof is in sphere.

### Embodiment 2

### Preparation of Fe-Based Particles with an Fe Elemental Core and Ag Coating Layer (Fe@Ag)

4.6 mM of ferrous sulfate and 0.46 mM of trisodium citrate dehydrate was mixed by stirring. The 8.8 mM of sodium borohydride was added into the mixture and stirred for another 10 min at room temperature. 0.05 M silver nitrate was further added into the mixture and then stirred for 5 min at room temperature in argon. The particles were then washed with ethanol for 3 times and collected via a magnet.

After the aforementioned process, Fe-based particles with Fe elemental cores and Ag coating layers as shells were obtained, in which the Ag coating layer was disposed on the whole surface of the Fe elemental core in each Fe-based particle. Herein, the size of each Fe-based particle is about 84.86±17.35 nm, the shape thereof is in sphere, and the thickness of the Ag coating layer is about 5 nm.

### Embodiment 3

### Preparation of Fe-Based Particles with Fe Elemental Core and Iron Oxide (Fe₂O₃) Shell (Fe@Iron Oxide)

To synthesize the Fe(iron oxide particles, 20 mL 1-octadecene and 0.3 mL oleylamine was mixed at 120° C. for 30 min in argon. Later, 0.7 mL iron pentacarbonyl was added to the 1-octadecene and oleylamine mixture at 180° C. for 20 min with continuously argon condition. The solution was cooled down to 160° C. before the further addition of 0.15 mL iron pentacarbonyl. Then, the solution was aged at 160° C. for 30 min with the presence of 0.3 mL oleic acid (1 mM). The particles were then washed with hexane and ethanol, and then kept in argon prior to the use. Herein, the iron oxide (Fe₂O₃) shell was formed spontaneously in a suitable environment, so the process for forming iron oxide shell can be omitted.

After the aforementioned process, Fe-based particles with Fe elemental cores and iron oxide coating layers as shells were obtained, in which the iron oxide coating layer was disposed on the whole surface of the Fe elemental core. In addition, the obtained particles were kept in argon, in order to prevent the Fe being oxidized. Herein, the size of each Fe-based particle is about 18.76±2.11 nm, and the shape thereof is in sphere.

### Cytotoxicity Assay (MTT Assay)

To evaluate the cytotoxicity of the obtained particles including Fe elemental particle (Embodiment 1), Fe@Ag (Embodiment 2) and Fe@Fe₂O₃ (Embodiment 3), cells growing in log-phase were seeded at a density of 5,000 cells per well in a 96-well culture plate. Prior to each experiment for administration in vitro, particles were resuspended in phosphate buffered saline solution (PBS) at 10 mg/mL as the stock solution. Cells were then treated by the assigned concentration for 48 hours. The 10×MTT stock (5 mg/mL) in PBS was diluted with culture medium as working solution. Cells were treated with the working solution for 1 hour. Later, the working solution was replaced by 50 μL DMSO to dissolve the violet crystal. The optical absorbance at 490 nm was measured in a microplate reader (Sunrise Absorbance Reader; Tecan, Miinnedorf, Switzerland). The cell viability was defined as: (O. D._{treated cells}−O. D._{DMSO blank})/(O. D._{untreated cells}−O. D._{DMSO blank})*100%.

### [Result]

### Evaluation on the Cytotoxicity of Fe-Based Particles Prepared in Embodiment 1

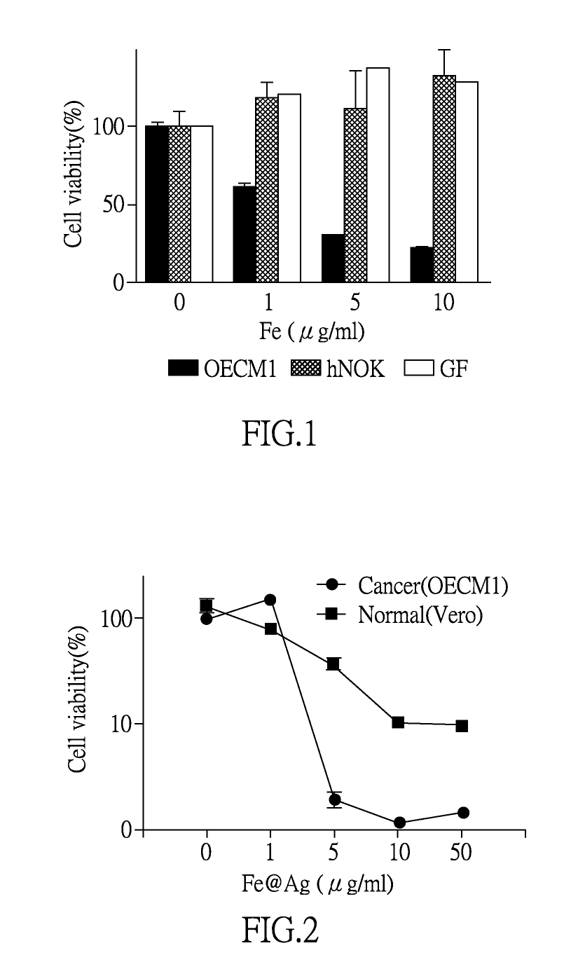

The cytotoxicity of Fe-based particles prepared in Embodiment 1 was evaluated by the aforementioned MTT assay. Herein, the cytotoxicity of Fe-based particles to OECM1 cell lines (tumor cells), human oral keratinocytes (hNOK, normal cells), human gingival fibroblast (GF, normal cells) was also evaluated by the aforementioned MTT assay. The result is shown in FIG. 1.

The X-axis of FIG. 1 is the addition amount of Fe-based particles, and the Y-axis thereof is the cell viability of cancer cells and normal cells treated with the Fe-based particles, in which the cell viability that the cells were not treated with the Fe-based particles was considered as 100%. As shown in FIG. 1, in the case that the cancer cells were treated with the Fe-based particles, the cell viability of cancer cells was about 20% when the dose of the Fe particles was about 10 μg/mL. However, even though the dose of the Fe particles was 10 μg/mL, the cell viability of normal cells was still over 100%. This result indicates that the Fe-based particles prepared in Embodiment 1 show killing selectivity to cancer cells, while sparing most of the normal cells.

### Evaluation on the Cytotoxicity of Fe-Based Particles Prepared in Embodiment 2 (Fe@Ag)

The cytotoxicity of Fe@Ag particles prepared in Embodiment 2 was evaluated by the aforementioned MTT assay. Herein, OECM 1 cell lines (cancer cells) and Vero cell lines (normal cells) were used. The result is shown in FIG. 2.

The X-axis of FIG. 2 is the addition amount of Fe@Ag particles, and the Y-axis thereof is the cell viability of cancer cells and normal cells, in which the cell viability that the cells were not treated with the Fe@Ag particles was considered as 100%. As shown in FIG. 2, in the case that the cancer cells were treated with Fe@Ag particles, the cell viability of cancer cells was about 5% when the dose of the Fe@Ag particles was more than 5 μg/mL. However, even though the dose of the Fe@Ag particles was 50 μg/mL, the cell viability of normal cells was still about 50%. This result indicates that the Fe@Ag particles prepared in Embodiment 1 show killing selectivity to cancer cells.

Evaluation on the Cytotoxicity of Fe-Based Particles Prepared in Embodiment 3 (Fe@Fe oxide)

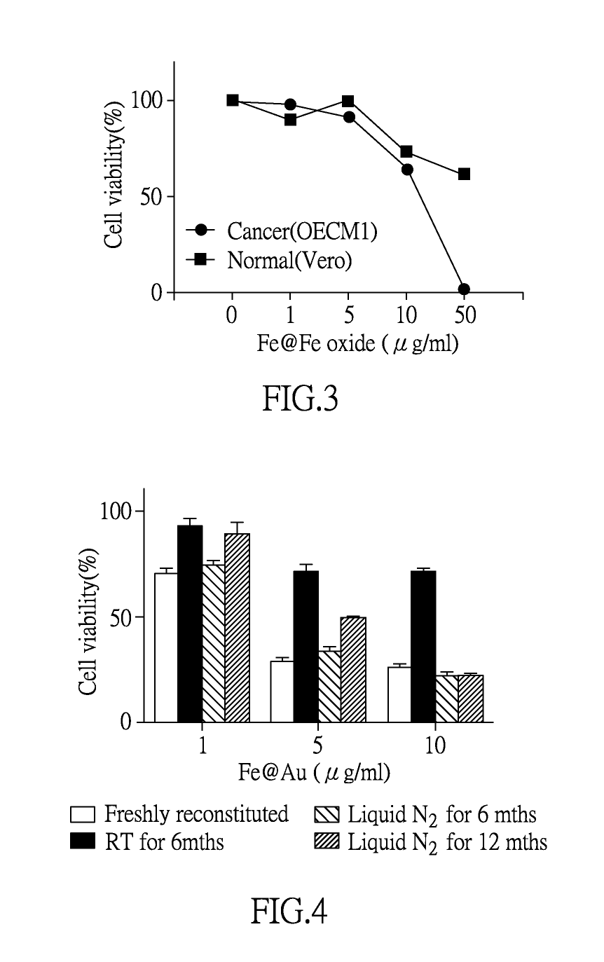

The cytotoxicity of Fe@Fe oxide particles prepared in Embodiment 3 was also evaluated by the aforementioned MTT assay. Herein, OECM 1 cell lines (cancer cells) and Vero cell lines (normal cells) were used. The result is shown in FIG. 3.

The X-axis of FIG. 3 is the addition amount of Fe@Fe oxide particles, and the Y-axis thereof is the cell viability of cancer cells and normal cells, in which the cell viability that the cells were not treated with the Fe@Fe oxide particles was considered as 100%. As shown in FIG. 3, in the case that the cancer cells were treated with Fe@Fe oxide particles, the cell viability of cancer cells was almost 0% when the dose of the Fe@Fe oxide particles was about 50 μg/mL. However, even though the dose of the Fe@Fe oxide particles was 50 μg/mL, the cell viability of normal cells was still about 60%. This result indicates that Fe@Fe oxide particles prepared in Embodiment 3 show killing selectivity to cancer cells, while sparing most of the normal cells.

### Evaluation on the Cytotoxicity of Fe@Au Nanoparticles

In the present experiment, Fe-based particles with Fe elemental core and Au covering layers as shells (Fe@Au) were used to evaluate the cytotoxicity thereof on OECM 1 cell lines by the aforementioned MTT assay. The result is shown in FIG. 4.

The X-axis of FIG. 4 is the addition amount of Fe@Au particles, and the Y-axis thereof is the cell viability of cancer cells and normal cells. As shown in FIG. 4, the cytotoxicity of Fe@Au particles shows dose-dependent. In addition, the Fe@Au particles stored in liquid N₂ still shows cytotoxicity as those freshly reconstituted Fe@Au particles. However, the cytotoxicity of Fe@Au particles stored at room temperature (RT) for 6 months is greatly reduced, compared to the freshly reconstituted Fe@Au particles. The reason for the reduced activity of the Fe@Au particles stored at room temperature (RT) for 6 months is due to the oxidation of the Fe elemental cores of the Fe-based particles.

This result indicate when the Fe elemental cores of the Fe-based particles were kept in zero valent irons, the activity thereof can still be reserved even though the Fe-based particles are placed for a long time.

In conclusion, the Fe-based particles with or without core-shell structures of the present invention show great killing selectivity to cancer cells, without any extra loaded drugs. In addition, when the Fe-based particles of the present invention stored in a suitable environment such as low-temperature condition, for example, liquid N₂, the activity of the Fe-based particles can be maintained for a long time. Hence, the Fe-based particles of the present invention can be prepared in advance, and then applied to tumor therapeutics. In addition, the Fe or Fe-based core-shell particles also have magneticity, so they can be applied to various diagnoses such as CT or MRI. Furthermore, with the selection of metal shell coated on the particles, it could achieve the specified diagnostic function. For example, the Ag shell on the Fe-based particles has the non-linear optical frequency multiplication enabled medical imaging application. Furthermore, the Pt shells of the Fe-based particles can also be used as contrast agents for CT imaging. Hence, the Fe-based particles of the present invention can be modulated based on the patient's symptom, the applications, the diagnosis methods, and the therapeutic methods.

Although the present invention has been explained in relation to its preferred embodiment, it is to be understood that many other possible modifications and variations can be made without departing from the spirit and scope of the invention as hereinafter claimed.

What is claimed is:

1. A method for treating a cancer, comprising:
administering an effective amount of Fe-based particles to a subject in need, wherein the Fe-based particles have core-shell structures, and each Fe-based particle comprises:
an Fe elemental core with zero valent irons; and
a covering layer formed on partial or whole surface of the Fe elemental core, wherein a material of the covering layer is a metal, a metal doped with dopants, a metal alloy, a polymer, carbon, a metal oxide or a nonmetal oxide, and the shape of the Fe-based particles is a rod, a sphere, a cubic or a dumbbell, with the proviso that the metal is not Au.

2. The method as claimed in claim 1, wherein the cancer is an oral cancer.

3. The method as claimed in claim 1, wherein the material of the covering layer is Ag, Pt, a metal oxide, an AgPt alloy, an AgAu alloy, or a PtAu alloy.

4. The method as claimed in claim 3, wherein the material of the covering layer is Ag.

5. The method as claimed in claim 3, wherein the metal oxide is ferric oxide.

6. The method as claimed in claim 1, wherein a size of each Fe-based particle is in a range from 5 nm to 5 μm.

7. The method as claimed in claim 6, wherein the size of each Fe-based particle is in a range from 5 nm to 1 μm.

8. The method as claimed in claim 7, wherein the size of each Fe-based particle is in a range from 5 nm to 50 nm.

9. The method as claimed in claim 1, wherein a size of the Fe elemental core in each Fe-based particle is in a range from 5 nm to 50 nm.

10. The method as claimed in claim 1, wherein a thickness of the covering layer in each Fe-based particle is in a range from 0.7 nm to 6 nm.

11. A method for treating a cancer, comprising:
administering an effective amount of Fe-based particles to a subject in need, wherein each Fe-based particles is an Fe elemental particle with zero valent irons.

12. The method as claimed in claim 11, wherein the cancer is an oral cancer.

13. The method as claimed in claim 11, wherein a size of each Fe-based particle is in a range from 5 nm to 5 μm.

14. The method as claimed in claim 13, wherein the size of each Fe-based particle is in a range from 5 nm to 1 μm.

15. The method as claimed in claim 14, wherein the size of each Fe-based particle is in a range from 5 nm to 50 μm.
